(19) United States
(12) Patent Application Publication
(10) Pub. No.: US 2010/0047919 A1
(43) Pub. Date: Feb. 25, 2010

(54) BIOSENSOR DEVICE AND METHOD FOR DETECTING MOLECULES IN AN ANALYTE

(75) Inventors: Derk Jan Wilfred Klunder, Eindhoven (NL); Maarten Marinus Johannes Wilhelms Van Herpen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/513,525

(22) PCT Filed: Nov. 6, 2007
(86) PCT No.: PCT/IB07/54486

§ 371 Date: May 5, 2009

(30) Foreign Application Priority Data
Nov. 10, 2006 (EP) 06123796.2

(51) Int. Cl. G01N 21/76; G01N 33/50

(52) U.S. Cl. 436/172; 422/52

(57) ABSTRACT

A biosensor device (10) for detecting molecules (16) in an analyte (14) comprises a binding element (12) which can be brought into contact with the analyte (14) for binding the molecules (16) thereto. The binding element (12) is an optical waveguide (34) comprising a strip (36) on a first layer (38). Light (17) can be applied to the strip (36). The biosensor device (10) further comprises an optical detection system (22) for detecting luminescent light (20) emitted by the excited molecules (16).

### FIELD OF THE INVENTION

The invention relates to a biosensor device for detecting molecules in an analyte.

The invention further relates to a method for detecting molecules in an analyte.

In general, a biosensor device is used to detect molecules in an environment by means of binding the molecules to the binding element of the biosensor device and measuring a change in chemical or physical properties of the biosensor device. In this way, the binding element is directly exposed to the environment of the biosensor device, e.g. to air or to an analyte comprising the molecules.

### BACKGROUND OF THE INVENTION

A biosensor device for detecting molecules in an analyte is known from US 2006/0045809 A1 that comprises a binding element designed as optical waveguide with a strip arranged on a substrate in a Mach-Zehnder interferometry configuration. The waveguide can be supplied with light, when the analyte is brought into contact with the binding element. The biosensor device further comprises oscillation excitation means to induce an oscillatory movement of those molecules bound to the strip. Due to this oscillatory movement, a phase difference of the supplied light between a waveguide portion with bound molecules and a reference waveguide portion without bound molecules is induced. The measured phase difference is converted into an output signal from which the amount of bound molecules is deduced.

Its is a disadvantage of this biosensor device, that the measurement principle as well as the design of this biosensor device are highly complicated, since much technical effort is required to realize this kind of interferometry measurement as well as the conversion and analysis of the output signal. Therefore the production and operating costs of this biosensor device are high compared to a biosensor device that is based on a simple detection mechanism.

In addition, it is a disadvantage of this biosensor device, that a quantitative analysis of bound molecules is rather sophisticated, as there is no direct correlation between the amount of bound molecules and the phase difference of the light measured in the Mach-Zehnder interferometry configuration. Thus further measurements of e.g. the oscillation frequency of receptor molecules with and without bound molecules are required causing additional technical effort for realizing such measurements.

Moreover, it is a disadvantage, that this biosensor device is limited to few specific applications, since the receptor molecules are limited to few specific species of molecules to be detected. In case of detecting other molecules, different receptor molecules have to be arranged on the strip.

### SUMMARY OF THE INVENTION

It is an object of the present invention to provide a biosensor device for detecting molecules in an analyte which allows for a very simple detection principle and which is highly sensitive to various species of molecules.

Further, it is an object of the present invention to provide a method for detecting molecules in an analyte.

According to the invention, this object is achieved by a biosensor device for detecting molecules in an analyte, comprising a binding element which can be brought into contact with the analyte for binding the molecules thereto, wherein the binding element is an optical waveguide comprising a strip arranged on a first layer, wherein light can be applied to the strip, further comprising an optical detection system for detecting luminescent light emitted by the excited molecules.

Further, according to the invention this object is achieved by a method for detecting molecules in an analyte, comprising the steps (a) providing a biosensor device according to the invention, (b) binding the molecules to the binding element, (c) applying light to the strip of the biosensor device, and (d) detecting luminescent light emitted by the excited molecules.

According to the invention, it is understood that the term “molecules” generally refers to molecules, beads and particles.

The biosensor device and the method provide a new concept for detecting molecules, particles and beads in an analyte. This biosensor device comprises a binding element in the form of an optical waveguide with a strip arranged on a first layer. When light is applied to the strip, TM polarized waveguide modes propagate through the strip. Here, TM polarization means that the electric field is parallel to the normal of the interface between the first layer and the strip. These waveguide modes evoke excitation light in a transverse direction of the propagation direction of the waveguide modes, i.e. transverse to the extension of the strip. The excitation light represents the evanescent field of the waveguide modes. It is concentrated around the strip and its intensity decays with the distance from the strip. The excitation light can be absorbed by the molecules near the binding element, i.e. near the strip, in order to get excited. According to the invention, “binding molecules to the binding element” means chemically binding the molecules to e.g. a surface of the strip. However, this expression also includes that diffusion causes the molecules to come into such a close proximity of the strip without coming in contact thereto, so that they get excited by the excitation light near the binding element. When returning to the not excited state, luminescent light, in particular fluorescent light, is emitted by the molecules and can be detected by an optical detection system. According to the invention, it is understood that the molecules either comprise autoluminescence properties or they are luminescent labeled with luminescent marker molecules for emitting the luminescent light. This concept of the biosensor device advantageously uses the excitation light provided by the strip waveguide to excite the molecules, so that no further means are necessary to cause a change in a chemical or physical property of the biosensor device that can be detected.

Moreover, the excitation light for the molecules is concentrated around the strip, so that the molecules are predominately excited when binding to the strip. In this way, background noise resulting from excited molecules or other excited impurities in the analyte away from the strip is significantly reduced. Furthermore, the highest optical intensity is at the interface between the strip and its environment, where also the binding sites are located. Thus, the biosensor device advantageously provides a very high detection efficiency and surface specificity for the molecules to be detected.

Advantageously, the biosensor device and the method for detecting molecules in an analyte further allows to identify the bound molecules as well as quantitatively determine the amount of bound molecules by means of detecting the luminescent light. Determining the wavelength of the luminescent light directly gives information about the transition between the not excited and excited states of the bound molecules and thus about the molecules themselves. The amount of bound molecules can be directly estimated from the measured intensity of the luminescent light.

In a preferred embodiment of the biosensor device, the binding element can be electrically charged in order to influence electrically charged molecules.

In this context, “influencing charged molecules” means attracting molecules which are oppositely charged with respect to the binding element as well as repulsing equally charged molecules from the binding element.

In this way, the binding element advantageously acts as electrode which can influence molecules based on their intrinsic charge. This offers a very easy way to manually set the sensitivity of the biosensor device. Moreover, using the charge of the molecules to be detected further enables the biosensor device to be used in many applications, as many molecules comprise free positive or negative charges. In particular, charging the binding element oppositely respecting the charge of the molecules to be detected offers a very simple possibility to improve the concentration of the molecules near the strip where the excitation light is located, since the molecules are attracted to the binding element based on their intrinsic electric charge. In turn, charging the binding element equally with respect to the electric charge of the molecules offers the possibility to reduce the background noise and to improve the signal to noise ratio of the biosensor device, since undesired molecules are moved away from the binding element, thus being not excited and detected anymore by the optical detection system. The latter effect can be used e.g. during a “washing step” that is performed after having bound numerous molecules to the binding element. During this procedure not bound molecules, which are still left in the analyte within reach of the evanescent field of the waveguide modes, are repulsed from the binding element, thus leaving the optical field and providing no luminescent signal. Furthermore, equally charging the binding element with respect to the charge of the molecules allows to distinguish between specifically and nonspecifically bound molecules, as specific bindings are usually stronger than nonspecific bindings. Nonspecifically bound molecules, i.e. molecules which are chemically bound to the binding element despite another desired binding reaction, are removed from the binding element by pulling them away with such a force which is sufficient to remove the nonspecific bindings and insufficient to remove the specific bindings.

In a further preferred embodiment of the biosensor device, the biosensor device further comprises a counterelement, wherein a charge of the binding element and an opposite charge of the counterelement can be alternately changed.

The advantage here is that the oppositely charged counterelement acts as counterelectrode for the binding element and molecules are thus moved along the electric flux lines between both. Alternately changing both the charge of the binding element and the charge of the counterelement which is opposite to the charge of the binding element improves the specific binding of the desired molecules, as these molecules are first attracted by the binding element and afterwards undesired molecules, i.e. not bound or nonspecifically bound molecules, are repulsed from the binding element. Therefore, the signal to noise ratio of the biosensor device is significantly increased. In this context, it is preferred that the change of the charges of the binding element and the counterelement is periodically performed. Here, the time period between the changes can be adapted to the desired application of the biosensor device.

According to an alternative preferred embodiment of the biosensor device, the binding element can be supplied with a current in order to influence magnetic molecules.

The advantage here is that the binding element provides a magnetic field, and intrinsic magnetic properties of the molecules, e.g. their susceptibility or their magnetic dipole moment, are used to actuate them and thus to control the detection sensitivity of the biosensor device, as the molecules in the analyte are attracted to and repulsed from the binding element. According to the invention, it is understood that the magnetic molecules either comprise intrinsic magnetic properties or they are labeled with magnetic molecules which have e. g. a susceptibility substantially different from the environment. For molecules with a susceptibility larger than the environment the molecules are attracted by the binding element, as the molecules experience a magnetic force towards regions with a higher magnetic intensity (cf. Moore at al., J. Magn. Mat. 225, 2001, 277-284). In turn, those molecules with a susceptibility smaller than the environment are repulsed from the binding element, as a magnetic force directing towards regions with smaller magnetic intensity acts on them.

In a further preferred embodiment, the biosensor device further comprises a counterelement, wherein the binding element and the counterelement can be alternately supplied with a current in order to influence the magnetic molecules.

The advantage here is that first desired magnetic molecules are attracted by the binding element for increasing the detection signal and afterwards undesired molecules are attracted by the counterelement and thus moved out of reach of the evanescent field leading to an improved signal to noise ratio of the biosensor device. The attraction of the magnetic molecules to the counterelement can be used similarly to the electric actuation e.g. during the washing step or to distinguish between specifically and nonspecifically bound molecules. Here, it is preferred that the supply of the current to the binding element and to the counterelement is periodically performed, where the time period between the change between both can be adapted to the desired application of the biosensor device. The counterelement for magnetically actuating the molecules to be detected can differ from the counterelement for electrically actuating the molecules.

In a further preferred embodiment of the biosensor device, the strip and the first layer are at least partially exposed to the analyte which forms a second layer for the optical waveguide.

Designing the optical waveguide in such a way advantageously enables a compact and economic biosensor device, where the analyte is used as second layer for the strip waveguide. Moreover, the surface specificity of the biosensor device can be controlled by setting the refractive index difference Δn between the first and the second layers. This index difference Δn is limited by the fact that in case of a too large asymmetry in the refractive indices n₁, n₂ between the first and second layers, the waveguide modes are below cut-off and not guided along the strip anymore.

In a further preferred embodiment of the biosensor device, the strip is made of a metal.

The advantage here is that metal is a conductive material, so that the metal strip can easily be electrically charged or supplied with a current without further means.

In a further preferred embodiment of the biosensor device, the metal strip is made of silver.

Silver shows a lower propagation loss of the waveguide modes propagating along the strip compared to other metals for a given width w and thickness d of the strip. Therefore, a longer length of the strip can be used to excite the molecules, or for a fixed distance to the point where light is coupled into the waveguide the power propagating through the waveguide is higher. Therefore, the efficiency of the biosensor device is advantageously increased. The metal strip can also be made of aluminum, gold or chromium.

In a further preferred embodiment of the biosensor device, the first layer is made of a dielectric material.

A dielectric layer acts as dielectric isolation of the metal strip, so that an electrical isolation of the metal strip to the environment is achieved. The dielectric layer can be made of a polymer or an oxide, for instance.

In a further preferred embodiment of the biosensor device, the refractive index n₂ of the analyte is adaptable to a refractive index n₁ of the first layer by setting a parameter of the analyte.

It is advantageous for the propagation loss of an optical waveguide, if its first and second layers show an almost symmetric structure regarding their refractive indices n₁, n₂ and thicknesses d. A symmetric structure is less sensitive for local variations in the layer thickness d, especially for a small layer thickness d, and in the refractive indices n₁, n₂, which could result in the mode being below cut-off and thus in a local leakage into the first and second layers. Setting a parameter of the analyte advantageously represents a very easy and well controllable way to achieve an index matching analyte for the first layer. In addition, this method of setting the refractive index n₂ of the analyte is very flexible in the sense that almost every required refractive index n₂ of the analyte can be achieved by changing an internal parameter of the analyte. For instance, in case of an aqueous sucrose solution as analyte the refractive index n₂ of the solution can be changed by simply adding sucrose to the solution.

In a further preferred embodiment of the biosensor device, the refractive indices n₁, n₂ of the first layer and the analyte are at least approximately equal.

It is known that for such a symmetric layer stack, where the refractive indices n₁, n₂ of the first and second layers are almost identical, there is no cut-off of the waveguide mode, as there is a guiding mode for every thickness of the strip. For a sufficiently small difference Δn between the refractive indices n₁, n₂, the waveguide mode is still reasonably far from cut-off and the light of the waveguide mode is substantially located around the strip. Therefore a substantially symmetric layer stack with a small difference Δn in the refractive indices n₁, n₂ of the first and second layers enables the waveguide being not really sensitive for propagation losses as a result of radiation of light into the first and/or second layers due to variations in the thickness d, which locally pushes the waveguide mode under cut-off. Furthermore, the decay lengths of the waveguide are small in both the first and second layers, resulting in a biosensor device with a high surface specificity for the molecules bound to this strip. It is preferred that the difference Δn in the refractive indices n₁, n₂ of the first and second layers is less than 0.004.

In a further preferred embodiment of the biosensor device, the refractive indices n₁, n₂ of the first layer and the analyte are approximately 1.45.

A first and second layer with such a refractive index offer in combination with a silver strip to which light of e.g. 600 nm wavelength is applied an optimum of excitation light around the strip, advantageously leading to a high efficiency of the biosensor device.

In a further preferred embodiment of the biosensor device, the first layer is transparent for excitation light and the analyte is transparent for the excitation light and luminescent light emitted by the molecules.

The advantage here is that the first and second layers being transparent for the excitation light of the molecules provide a homogeneous excitation light distribution around the strip which can be used to excite molecules near the strip. A further transparency of the analyte for the luminescent light enables the luminescent light to be detected outside the analyte, so that the detection system for the luminescent light can be located e.g. next to the analyte.

In a further preferred embodiment of the biosensor device, a thickness d of the strip is between 1 nm and 10 nm, preferably between 3 nm and 8 nm, still preferably between 5 nm and 6 nm.

With thicknesses d of the strip in these ranges the propagation losses along the strip can be kept reasonably small and in turn a homogenous excitation light of sufficient intensity is obtained, which advantageously leads to an increased excitation efficiency for the molecules. With decreasing thickness d, the propagation losses also decrease. The optimum thickness d depends on the application of the biosensor device and represents a compromise between the propagation losses and the desired decay lengths of the waveguide modes into the first and second layers.

In a further preferred embodiment of the biosensor device, a width w of the strip is between 2 μm and 5 μm.

The advantage here is that a strip width w of such values leads to a sufficient small propagation loss, thereby providing a sufficient large area of the strip which is exposed to the analyte and which is thus useful for the detection of the molecules. It is possible that the strip comprises along its extension sections with different widths w, such that those sections with broader widths w result in a higher output signal of the biosensor device, as more molecules can bind to the metal strip.

In a further preferred embodiment of the biosensor device, an adhesion layer for binding the molecules is formed on top of the strip.

An adhesion layer on top of the strip advantageously increases the sensitivity and specificity of the biosensor device to certain species of molecules, since the adhesion layer may comprise receptor molecules to which only certain species of molecules can bind.

In a further preferred embodiment of the biosensor device, the optical detection system for detecting the luminescent light is located next to the analyte.

The advantage here is that the luminescent light emitted by the molecules can be received by the detection system without substantial losses, as the detection system is located near the second layer which is transparent for the luminescent light emitted by the molecules.

In a further preferred embodiment of the biosensor device, the strip, the first layer, the analyte and the optical detection system are arranged in a housing.

Advantageously, the biosensor device is constructed in such a way that all essential units are implemented into the housing. In this way, the biosensor device can be used in a flexible way outside e.g. a laboratory at various locations.

Further advantages will be apparent from the following description and the accompanying drawings.

It is to be understood that the afore-mentioned features and those to be explained below are not only applicable in the combinations given, but also in other combinations or in isolation without departing from the scope of the invention.

### BRIEF DESCRIPTION OF THE DRAWINGS

An exemplary embodiment of the invention is illustrated in the drawings and will be described hereinafter with reference thereto. In the drawings:

FIG. 1A is a schematic sectional view of a biosensor device in accordance with the invention;

FIG. 1B is another schematic sectional view of the biosensor device in FIG. 1A;

FIG. 2 is a diagram of an attenuation of a fundamental waveguide mode depending on a thickness d of a slab waveguide;

FIG. 3 is a diagram of an effective refractive index n_{eff} of TM polarized waveguide modes depending on a width w of a strip waveguide;

FIG. 4 is a diagram of the attenuation of the TM polarized waveguide modes depending on the width w of a strip waveguide;

FIG. 5 is a diagram of a decay length of the fundamental waveguide mode in first and second layers of a slab waveguide depending on a difference Δn in refractive indices n₁, n₂ of the first and second layers;

FIG. 6 is a diagram of the refractive index n₂ of an aqueous sucrose solution, depending on a sucrose concentration in the solution.

### DETAILED DESCRIPTION OF SOME EMBODIMENTS OF THE INVENTION

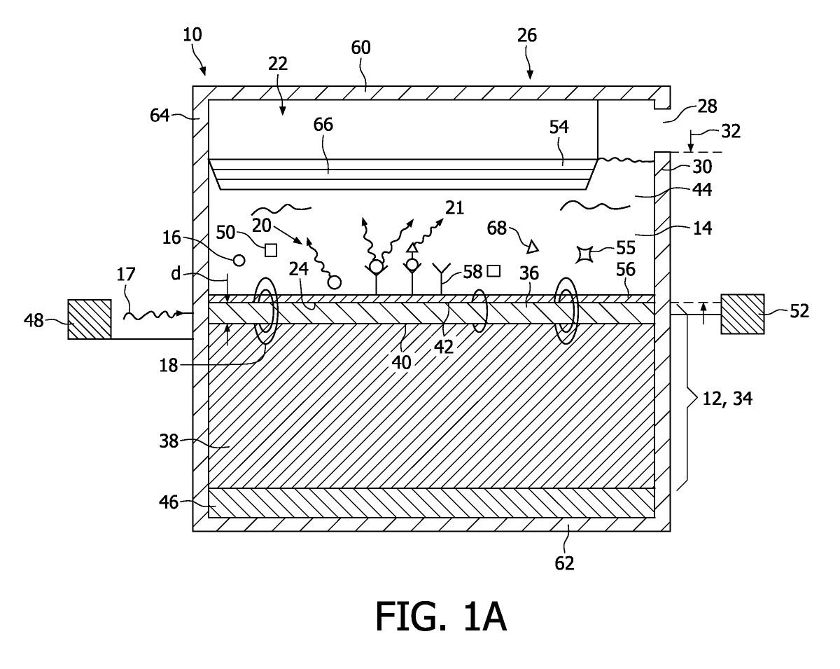

FIGS. 1A and 1B show a biosensor device 10 in a schematic representation. The biosensor device 10 is used to detect molecules such as biomolecules like DNA in an analyte, where the detection sensitivity of the biosensor device 10 can be increased based on the magnetic or electric properties of the molecules to be detected.

According to the invention, the term “molecules” generally refers to molecules, beads and particles which are comprised in the analyte.

The biosensor device 10 comprises a binding element 12 that can be brought into contact with the analyte 14 for binding the molecules 16 in the analyte 14. Diffusion causes the molecules in the analyte 14 to move towards and away from the binding element 12 for binding to the binding element 12. Light 17 is applied to the binding element 12 providing excitation light 18 for the molecules 16 located around the binding element 12, as the excitation light 18 is concentrated around the binding element 12. Upon binding to the binding element 12, the molecules 16 are excited by the excitation light 18 and they emit luminescent light 20, in particular fluorescent light 21. The luminescent light 20, here the fluorescent light 21, is an output signal of the biosensor device 10 and it is detected by an optical detection system 22.

Accordingly to the invention, “binding molecules 16 to the binding element 12” means in this context chemically binding the molecules 16 to e.g. a surface 24 of the binding element 12 or coming into such a close proximity of the binding element 12 without coming in contact thereto, so that the excitation light 18 near the binding element 12 excites the molecules 16.

The biosensor device 10 comprises a housing 26 made of a non-conductive and transparent material such as glass, in which the binding element 12 is located. The analyte 14 can be filled into the housing 26 using a pipette via an opening 28 which is located on a side wall 30 of the housing 26 in such a height 32 that a sufficient amount of the analyte 14 can be put into the housing 26 on top of the binding element 12. The opening 28 can also be located on top of the glass housing 26.

The binding element 12 is designed as an optical waveguide 34 comprising a thin, in particular ultra thin strip 36 (thickness d, width w) arranged on a first layer 38. Preferably, the strip 36 is located on top of a surface 40 of the first layer 38. However, the strip 36 can also be embedded into the first layer 38 in such a way that the surface 40 of the first layer 38 essentially coincides with an upper surface 42 of the strip 36. The strip 36 can comprise sections with different widths w, such that the detection efficiency of the biosensor device 10 may vary along the extension of the strip 36. The strip 36 is further made of a metal, whereas the first layer 38 is made of a dielectric material with a refractive index n₁ of approximately 1.45. For instance, the first layer 38 is made of a polymer or an oxide.

The surface 42 of the metal strip 36 as well as the surface 40 of the first layer 36 are at least partially exposed to the analyte 14 which forms a second layer 44 for the optical waveguide 34. Both the first and the second layers 38, 44 are transparent for the excitation light 18, whereas the second layer 44 is also transparent for the fluorescent light 21 emitted by the molecules 16. The analyte 14 is an aqueous sucrose solution whose refractive index n₂ is at least approximately equal to the refractive index n₁ of the first layer 38.

The biosensor device 10 further comprises a substrate 46 supporting the first layer 38. The substrate 46 can be made of silica.

The excitation light 18 for the molecules 16 is provided by the optical waveguide 34. When applying the light 17 of a light source 48, e.g. of a laser, that is located outside the housing 28 of the biosensor device 10, to the strip 36, TM polarized waveguide modes are transferred through the metal strip 36. These TM polarized waveguide modes propagate along interfaces between the strip 36 and the first and second layers 38, 44 evoking the excitation light 18 as evanescent fields of the waveguide modes in the first and second layers 38, 44 in a transverse direction compared to a propagation direction of the waveguide modes. The excitation light 18 is concentrated around the metal strip 36 and its intensity essentially decays with the distance from the strip 36. Therefore mostly molecules 16 near the strip 36 are excited and emit the fluorescence light 21, and background signals resulting e.g. from an excitation of the molecules 16 far away from the strip 36 or impurities 50 in the analyte 14 are significantly reduced.

In order to increase the sensitivity of the biosensor device 10, the binding element 12 can be electrically charged in order to influence and actuate electrically charged molecules 16. Therefore the biosensor device comprises means 52 to apply a voltage to the binding element 12 and to a metal counterelement 54 which is spaced to the binding element 12 and located next to and in contact with the analyte 14. These means 52 are located outside the housing 26 of the biosensor device 10 and can be designed as a conventional power source. The binding element 12 and the counterelement 54 are oppositely charged and act as electrode and counterelectrode. The electrically charged molecules 16 thus move along electric flux lines between the binding element 12 and the counterelement 54.

When charging the binding element 12, i.e. the metal strip 36, oppositely and the counterelement 54 equally with respect to a charge of the molecules 16 to be detected, the molecules 16 are attracted to the binding element 12 based on their intrinsic electric charge. For example, negatively charged DNA molecules 16 are attracted by a positively charged binding element 12. In turn, charging the binding element 12 equally with respect to the electric charge of the molecules 16 and thus the counterelement 54 oppositely results in undesired molecules 16 moving away from the binding element 12, thus being not excited and detected anymore by the optical detection system 22. The latter kind of charging the binding element 12 and the counterelement 54 can be used e.g. for a “washing step” that is performed after numerous molecules 16 have bound to the binding element 12. During this procedure not bound molecules 16, which are still left in the analyte 14 within reach of the evanescent field of the waveguide modes, are repulsed from the binding element 12, thus leave the optical field and provide no luminescent or fluorescent signal. Furthermore, this way of charging the binding element 12 and the counterelement 54 allows to distinguish between specifically and nonspecifically bound molecules 16, as specific bindings are usually stronger than nonspecific bindings. Nonspecifically bound molecules 16, i.e. molecules 16 which are chemically bound to the binding element 12 despite another desired binding reaction, are removed from the binding element 12 by pulling them away with such a force which is sufficient to remove the nonspecific bindings and insufficient to remove the specific bindings.

Furthermore, the binding element 12 and the counterelement 54 can be alternately charged, so that first molecules 16 to be detected are attracted by the binding element 12 and afterwards undesired molecules 16, such as not bound molecules 16 or those with nonspecific bindings, are attracted by the counterelement 54. Therefore the efficiency, the sensitivity and the specifity of the biosensor device 16 are significantly increased. The alternation of the voltage application to the binding element 12 and the counterelement 54 is periodically performed, wherein the time interval between the voltage changes can be adapted to e.g. the kind of molecules 16 to be detected.

Alternatively, a current can be supplied to the metal strip 36 using the means 52 resulting in a magnetic field around the metal strip 38 which influences the molecules 16 in the analyte 14 based on their intrinsic magnetic properties resulting from e.g. their magnetic dipole moment or their susceptibility. In case the molecules 16 to be detected do not comprise any intrinsic magnetic properties, it is understood that the molecules 16 can be labeled with magnetic molecules 55 which have e. g. a susceptibility substantially different from the environment. Molecules 16 with a susceptibility larger than the environment are attracted by the binding element 12, as these molecules 16 experience a magnetic force towards regions with a higher magnetic intensity. In contrast, those molecules 16 with a susceptibility smaller than the environment are repulsed from the binding element 12, as a magnetic force directing towards regions with smaller magnetic intensity act on them.

Furthermore, the counterelement 54 can be supplied with a current using the means 52. The attraction of the magnetic molecules 16 to it can be used similarly to the electric actuation e.g. during the washing step or to distinguish between specifically and nonspecifically bound molecules. Alternately supplying a current to the binding element 12 and the counterelement 54 leads to an increased sensitivity and selectivity of the biosensor device 10. Here, it is preferred that the supply of the current to the binding element 12 and to the counterelement 54 is periodically performed, where the time period between the change can be adapted to the desired application of the biosensor device 10.

An adhesion layer 56 is further formed on top of the metal strip 36, i.e. on the upper surface 42 of the metal strip 36 that is exposed to the analyte 14. This adhesion layer 56 comprises receptor molecules 58 for binding at least a monolayer of the molecules 16, since the receptor molecules 58 interact with the molecules 16 to be detected and form a chemical bond between them. Therefore a selectivity for a special species of the molecules 16 is achieved and the impurities 50 in the analyte 14 cannot bind to the binding element 12.

The optical detection system 22 is located at an upper wall 60 of the housing 26 of the biosensor device 10 and it is received within the counterelement 54. In order to allow filling the analyte 14 into the housing 26, both the optical detection system 22 and the counterelement 54 are spaced from the side wall 30 of the housing 26. The optical detection system 22 can also be arranged between the substrate 46 and a lower wall 62, at one of the side walls 30, 64 of the housing 26 or outside the housing 26 or at combinations thereof. As the emitted fluorescent light 21 cannot penetrate through the metal film 36, it is preferred that the detection system 22 is located next to the analyte 14 at the upper wall 60. The optical detection system 22 can be designed as photodiodes 66 which convert the fluorescent light 21 into a current to be measured. This conversion allows for a quantitative analysis of the number of the excited molecules 16 in the analyte 14. Moreover, based on the wavelength of the emitted fluorescent light 21 the species of the bound molecules 16 can be determined. Therefore, the biosensor device 10 is arranged to quantitatively determine the molecules 16 in the analyte 14 as well as identify the molecules 16 themselves.

Assuming the molecules 16 not acting as luminophores owing to a missing autofluorescence capability, they can be fluorescence labeled by adding fluorescence marker molecules 68 to the analyte 14. These marker molecules 68 chemically bind to the molecules 16 and emit the fluorescent light 21 when the molecules 16 with the marker molecules 68 bind to the binding element 12. Depending on the chemical structure of the molecules 16, only specific marker molecules 68 can form a chemical bond with the molecules 16. The fluorescence light 21 emitted from these marker molecules 68 helps to identify the original molecules 16.

The design of the optical waveguide 34 crucially determines the propagation capability of the light 17 through the waveguide 34 and, thus, the intensity of the excitation light 18 around the strip 36. The influence of the strip material, the thickness d and the width w of the strip 36 as well as the refractive indices n₁, n₂ of the first layer 38 and the second layer 44 are shown in the following analysis in which a wavelength of the TM polarized light 17 is approximately 600 nm (visible orange light), and the refractive indices n₁, n₂ of the first and second layers 38, 44 are approximately 1.45.

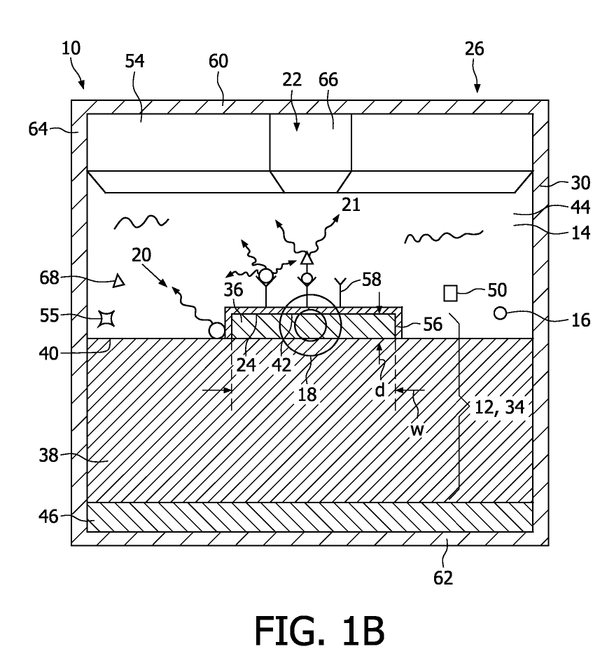

As shown in FIG. 2, the material of the metal strip 36 decisively determines an attenuation of a fundamental waveguide mode when passing through the strip 36. For a given thickness d of a metal slab waveguide, i.e. a thin strip with infinite width w, e.g. for d=5 nm, the attenuation of the fundamental slab mode ranges from 20 dB/cm to 120 dB/cm depending on the slab material, implying that a power of the guided slab mode drops to 10% of an input power after a distance of 0.8-5 mm. Thus, the thickness d of the metal strip 36 is preferably between 1 nm and 10 nm, further preferably between 3 and 8 nm, and further preferably between 5 nm and 6 nm. In addition, silver is shown to exhibit the lowest attenuation of the fundamental slab mode. Thus, the metal strip 36 is preferably made of silver, however, other materials such as gold (Au), aluminum (Al) and chromium (Cr) can be also used as strip material.

In the further analysis, a wavelength dependent complex refractive index nₘ=0.12+3.73i is used for the silver waveguide.

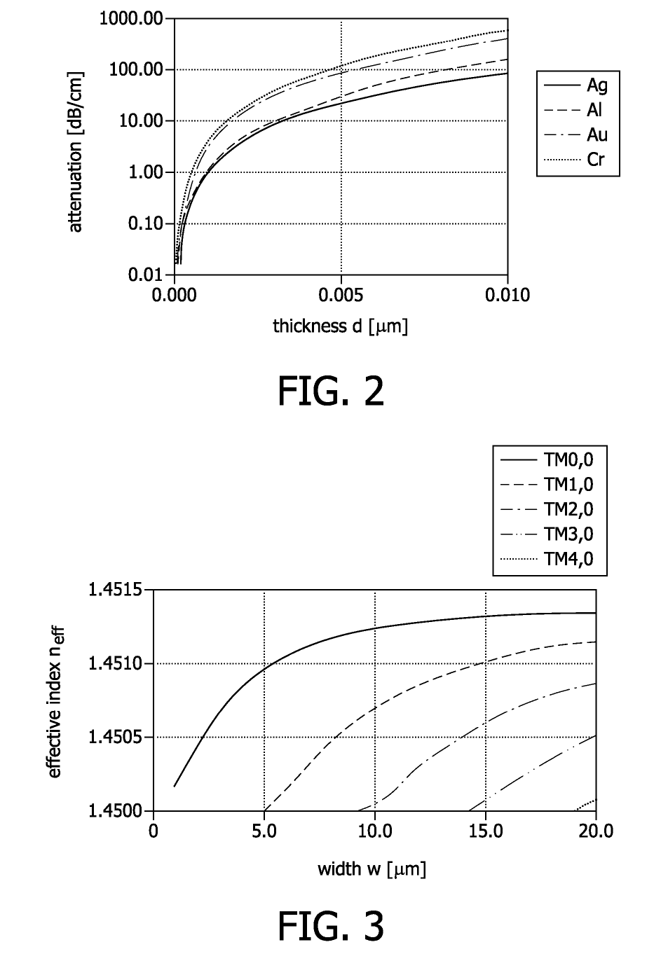

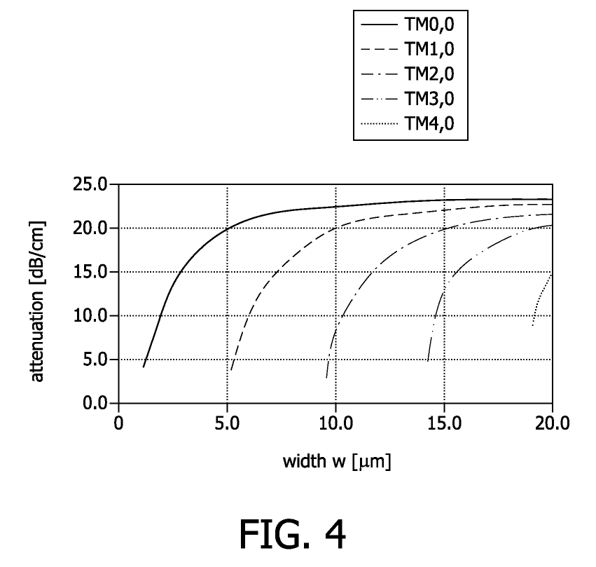

The propagation of the waveguide modes depends on the width w of the strip 36. The dependence of the effective index n_{eff} of the TM polarized waveguide modes TM_{p,q} on the width w of the silver strip 36 (d=5.1 nm) is shown in FIG. 3, where p and q denotes a lateral and vertical mode-order, respectively. An increasing width w of the silver strip 36 leads to an increasing effective index n_{eff} in the range of 1.4500 to 1.4515, and the multimode regime sets in above the strip width w of approximately 5.0 μm (see FIG. 4). The attenuation of the TM polarized waveguide modes TM_{p,q} of the silver strip 36 (d=5.1 nm) also increases with the width w of the strip. It is therefore preferred that the width w of the strip 36 is between 2 μm and 5 μm.

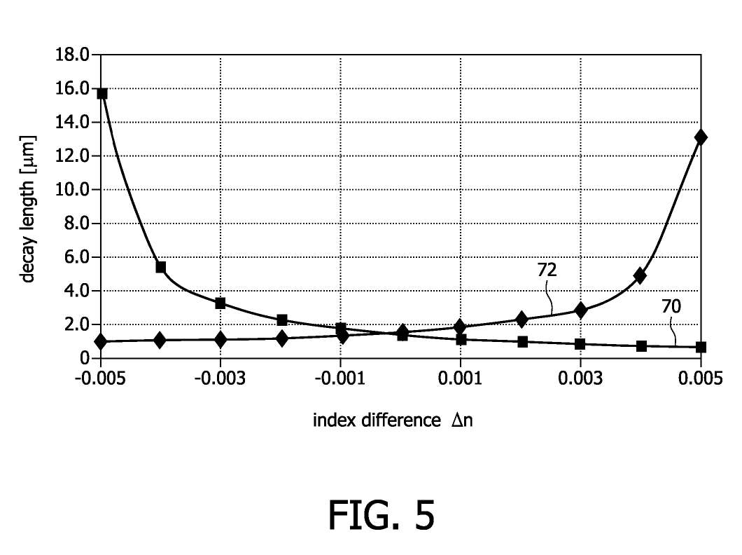

In order to support the waveguide mode propagation along the metal strip 36, the refractive indices n₁, n₂ of the first layer 38 and the second layer 44, i.e. the analyte 14, are approximately equal providing an almost symmetric layer stack. FIG. 5 shows a decay length ((1/e)² value of intensity) 70, 72 of the intensity of the fundamental TM waveguide mode of the first and second layers 38, 44 in case of a silver slab (d=5.1 nm) depending on the index difference Δn of the first and second layers 38, 44. For an index difference Δn of the first and second layers 38, 44 in the range of approximately −0.004 to +0.004, the decay length 70, 72 is smaller than 6 μm. A too large decay length 70, 72 could result in a leakage of the waveguide mode into the substrate 46 that supports the first layer 38, which implies a very thick second layer 44 to avoid this leakage. For instance, in case of a too thin second layer 44, the impact of the asymmetry between the first and the second layers 38, 44 induced by an air layer on top of the second layer 44 is too large, and the waveguide cannot support the waveguide modes anymore.

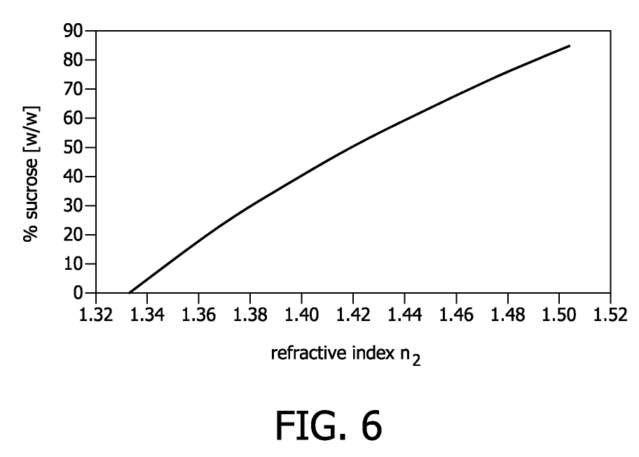

To achieve approximately equal refractive indices n₁, n₂ of the first and second layers 38, 44, the refractive index n₂ of the second layer 44, i.e. the analyte 14, is adaptable to the refractive index n₁ of the first layer 38 by setting a parameter of the analyte 14. In case of the aqueous sucrose solution, a sucrose concentration can be used to control the refractive index n₂ of the solution by adding small amounts of sucrose. FIG. 6 shows the dependence of the aqueous sucrose concentration on the refractive index n₂ of the solution at 20° C. Adding sucrose to the solution results in an approximately linear increase of the refractive index n₂ from 1.33 to 1.50.

It is possible that the biosensor device 10 comprises a plurality of metal strips 36, which are at least 1 μm spaced to each other. Thus, the output signal is increased as more analyte 14 can be brought into contact with the metal strips 36, thus more molecules 16 can be excited by the excitation light 18. This is particular advantageous, if only few molecules 16 are comprised in the analyte 14.

1. A biosensor device for detecting molecules (16) in an analyte (14), comprising a binding element (12) which can be brought into contact with the analyte (14) for binding the molecules (16) thereto, wherein the binding element (16) is an optical waveguide (34) comprising a strip (36) arranged on a first layer (38), wherein light (17) can be applied to the strip (36), further comprising an optical detection system (22) for detecting luminescent light (20) emitted by the excited molecules (16).

2. The biosensor device of claim 1, wherein the binding element (12) can be electrically charged in order to influence electrically charged molecules (16).

3. The biosensor device of claim 1, further comprising a counterelement (54), wherein a charge of the binding element (12) and an opposite charge of the counterelement (54) can be alternately changed.

4. The biosensor device of claim 1, wherein the binding element (12) can be supplied with a current in order to influence magnetic molecules (16).

5. The biosensor device of claim 1, further comprising a counterelement (54), wherein the binding element (12) and the counterelement (54) can be alternately supplied with a current in order to influence the magnetic molecules (16).

6. The biosensor device of claim 1, wherein the strip (36) and the first layer (38) are at least partially exposed to the analyte (14) which forms a second layer (44) for the optical waveguide (34).

7. The biosensor device of claim 1, wherein the strip (36) is made of a metal.

8. The biosensor device of claim 7, wherein the strip (36) is made of silver.

9. The biosensor device of claim 1, wherein the first layer (38) is made of a dielectric material.

10. The biosensor device of claim 1, wherein the refractive index n2 of the analyte (14) is adaptable to a refractive index n1 of the first layer (38) by setting a parameter of the analyte (14).

11. The biosensor device of claim 1, wherein the refractive indices n1, n2 of the first layer (38) and the analyte (14) are at least approximately equal.

12. The biosensor device of claim 1, wherein the refractive indices n1, n2 of the first layer (38) and the analyte (14) are approximately 1.45.

13. The biosensor device of claim 1, wherein the first layer (38) is transparent for excitation light (18) and the analyte (14) is transparent for the excitation light (18) and luminescent light (20) emitted by the molecules (16).

14. The biosensor device of claim 1, wherein a thickness d of the strip (36) is between 1 nm and 10 nm.

15. The biosensor device of claim 1, wherein the thickness d of the strip (36) is between 3 nm and 8 nm.

16. The biosensor device of claim 1, wherein the thickness d of the strip (36) is between 5 nm and 6 nm.

17. The biosensor device of claim 1, wherein a width w of the strip (36) is between 2 μm and 5 μm.

18. The biosensor device of claim 1, wherein an adhesion layer (56) for binding the molecules (16) is formed on top of the strip (36).

19. The biosensor device of claim 1, wherein the optical detection system (22) for detecting the luminescent light (20) is located next to the analyte (16).

20. The biosensor device of claim 1, wherein the strip (36), the first layer (38), the analyte (14) and the optical detection system (22) are arranged in a housing (26).

21. A method for detecting molecules (16) in an analyte (14), comprising the steps of:
(a) providing a biosensor device (10) of claim 1,
(b) binding the molecules (16) to the binding element (12),
(c) applying light to the strip (36) of the biosensor device (10),
(d) detecting luminescent light (20) emitted by the excited molecules (16).
